(19) United States
(12) Patent Application Publication
(10) Pub. No.: US 2025/0288792 A1
(43) Pub. Date: Sep. 18, 2025

(54) Cardiac Pump With Speed Adapted for Ventricle Unloading

(71) Applicant: TC1 LLC, St. Paul, MN (US)

(72) Inventors: Alexander Medvedev, Ann Arbor, MI (US); Masamichi Yanai, Ann Arbor, MI (US)

(73) Assignee: TC1 LLC, St. Paul, MN (US)

(21) Appl. No.: 19/225,561

(22) Filed: Jun. 2, 2025

Related U.S. Application Data
(63) Continuation of application No. 18/216,925, filed on Jun. 30, 2023, now Pat. No. 12,343,517.
Application No. 18/216,925 is a continuation of application No. 17/205,511, filed on Mar. 18, 2021, now Pat. No. 11,724,094.
Application No. 17/205,511 is a continuation of application No. 16/577,242, filed on Sep. 20, 2019, now Pat. No. 10,980,928.
Application No. 16/577,242 is a continuation of application No. 15/640,001, filed on Jun. 30, 2017, now Pat. No. 10,456,513.
Application No. 15/640,001 is a continuation of application No. 13/873,551, filed on Apr. 30, 2013, now Pat. No. 9,713,663.

(51) Int. Cl. A61M 60/232; A61M 60/148; A61M 60/178; A61M 60/515; A61M 60/538

(52) U.S. Cl. CPC A61M 60/232; A61M 60/178; A61M 60/515; A61M 60/538; A61M 60/148; A61M 2205/3334

(57) ABSTRACT

A blood pump system is implantable in a patient for ventricular support. A pumping chamber has an inlet for receiving blood from a ventricle of the patient. An impeller is received in the pumping chamber. A motor is coupled to the impeller for driving rotation of the impeller. A motor controller is provided for tracking systolic and diastolic phases of a cardiac cycle of the patient and supplying a variable voltage signal to the motor in a variable speed mode to produce a variable impeller speed linked to the cardiac cycle. The impeller speed comprises a ramping up to an elevated speed during the diastolic phase in order to reduce a load on the ventricle at the beginning of the systolic phase.

### CROSS-REFERENCES TO RELATED APPLICATIONS

The present application is a Continuation of U.S. patent application Ser. No. 18/216,925 filed Jun. 30, 2023 (Allowed); which is a Continuation of U.S. patent application Ser. No. 17/205,511 filed Mar. 18, 2021 (now U.S. Pat. No. 11,724,094); which is a Continuation of U.S. patent application Ser. No. 16/577,242 filed Sep. 20, 2019 (now U.S. Pat. No. 10,980,928); which is a Continuation of U.S. patent application Ser. No. 15/640,001 filed Jun. 30, 2017 (now U.S. Pat. No. 10,456,513); which is a Continuation of U.S. patent application Ser. No. 13/873,551 filed Apr. 30, 2013 (now U.S. Pat. No. 9,713,663).

### BACKGROUND OF THE INVENTION

The present invention relates in general to ventricular support pumps and controls, and, more specifically, to a ventricular assist device for reducing load applied to a weakened ventricle during the systolic phase.

Many types of circulatory assist devices are available for either short term or long term support for patients having cardiovascular disease. For example, a heart pump system known as a left ventricular assist device (LVAD) can provide long term patient support with an implantable pump associated with an externally-worn pump control unit and batteries. The LVAD improves circulation throughout the body by assisting the left side of the heart in pumping blood. One such system is the DuraHeart® LVAS system made by Terumo Heart, Inc., of Ann Arbor, Michigan. The DuraHeart® system employs a centrifugal pump with a magnetically levitated impeller to pump blood from the left ventricle to the aorta. The impeller can act as a rotor of an electric motor in which a rotating magnetic field from a multiphase stator couples with the impeller and is rotated at a speed appropriate to obtain the desired blood flow through the pump.

A typical cardiac assist system includes a pumping unit, drive electronics, microprocessor control unit, and an energy source such as rechargeable batteries and/or an AC power conditioning circuit. The system is implanted during a surgical procedure in which a centrifugal pump is placed in the patient's chest. An inflow conduit is pierced into the left ventricle to supply blood to the pump. One end of an outflow conduit is mechanically fitted to the pump outlet and the other end is surgically attached to the patient's aorta by anastomosis. A percutaneous cable connects to the pump, exits the patient through an incision, and connects to the external control unit. An LVAD system may be used with or without a pacemaker.

A control system for varying pump speed to achieve a target blood flow based on physiologic conditions is shown in U.S. Pat. No. 7,160,243 issued Jan. 9, 2007, which is incorporated herein by reference in its entirety. A target blood flow rate may be established based on the patient's heart rate so that the physiologic demand is met. In one type of conventional control unit, a constant speed setpoint has been determined for the pump motor to achieve the target flow based on demand. In this type of system, the pump speed is substantially constant, within an individual cardiac cycle.

Pulsatile pumping systems are also known wherein the pump speed is varied within the cardiac cycle to more closely mimic natural heart action. In one example, U.S. Pat. No. 8,096,935 to Sutton et al. oscillates the speed of the pump to produce a pulsed pressure. The speed is oscillated synchronously with the natural cardiac cycle such that a pump speed is increased during systole (the time of highest flow) and decreased during diastole (the time of lowest flow).

Whether operated at a constant speed or in a pulsatile manner, it is known that when desiring to obtain a maximum unloading of a weakened ventricle the average pump speed should be increased as much as possible (so that the pump flow is increased to the point where it captures almost the entire cardiac output). Due to flow inertia, however, the pump flow lags the ventricular pressure increase occurring at the beginning of systole. Therefore, the ventricle contraction still remains isometric at the beginning of systole (i.e., the pressure inside the ventricle resists its contraction). Furthermore, an increased average pump speed increases the risk of ventricular suction, particularly at the end of systole when the ventricle could be nearly empty.

### BRIEF SUMMARY OF THE INVENTION

In order to make ventricular contraction easier, the pump speed is increased before the systolic phase of cardiac cycle. As a result, the intra-ventricular pressure is reduced prior to ventricular contraction allowing a weak ventricle to contract with reduced resistance. In order to prevent ventricular suction, the pump speed is reduced before the end of systole when the ventricle is nearly empty.

In one aspect of the invention, a blood pump system is provided for implanting in a patient for ventricular support. A pumping chamber has an inlet for receiving blood from a ventricle of the patient. An impeller is received in the pumping chamber. A motor is coupled to the impeller for driving rotation of the impeller. A motor controller is provided for tracking systolic and diastolic phases of a cardiac cycle of the patient and supplying a variable voltage signal to the motor in a variable speed mode to produce a variable impeller speed linked to the cardiac cycle. The impeller speed comprises a ramping up to an elevated speed during the diastolic phase in order to reduce a load on the ventricle at the beginning of the systolic phase. In some embodiments, the impeller speed also comprises a ramping down to a reduced speed during the systolic phase to avoid collapse of the ventricle.

The variable speed mode may be comprised of a constant current mode or may be comprised of a speed control for matching impeller speed to a target speed in which the target speed ramps up to the elevated speed during the diastolic phase and ramps down to a reduced speed during the systolic phase to avoid collapse of the ventricle.

The motor controller may be configurable to provide the variable voltage signal to the motor in either the above variable speed mode or a constant speed mode. The constant speed mode maintains a substantially constant speed of the impeller over each respective cardiac cycle. A selection between the variable speed mode and the constant speed mode is determined according to a physiologic capability of the patient. This allows for selective therapy during LVAD support. For example, immediately following the implantation when the left ventricle is weak, the pump is set to operate in the constant current mode thereby providing a greater level of ventricle unloading. With the patient's recovery, the pump may be set to operate in the constant speed mode, promoting higher flow pulsatility and a more natural physiologic response to the patient's activities.

### BRIEF DESCRIPTION OF THE DRAWINGS

FIG. 1 is a diagram of a circulatory assist system as one example of an implantable pump employing the present invention.

FIG. 2 is an exploded, perspective view of a portion of a centrifugal pump of a type that can be used in the present invention.

FIG. 3 is a diagram showing a prior art variable speed profile synchronized with a cardiac cycle.

FIG. 4 is a diagram showing a variable speed profile of the present invention.

FIG. 5 is a diagram showing a variable speed profile in greater detail.

FIG. 6 is a block diagram showing a pump motor and control system of the present invention.

FIG. 7 is a diagram showing pump load and speed according to an embodiment using a constant current mode.

### DETAILED DESCRIPTION OF THE INVENTION

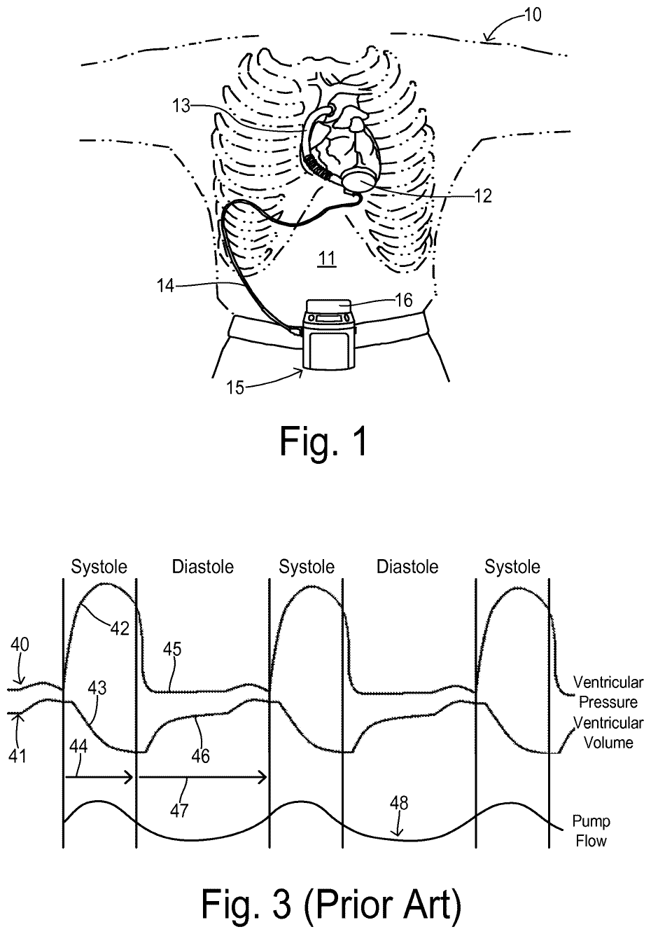

Referring to FIG. 1, a patient 10 is shown in fragmentary front elevational view. Surgically implanted either into the patient's abdominal cavity or pericardium 11 is the pumping unit 12 of a ventricular assist device. An inflow conduit (on the hidden side of unit 12) pierces the heart to convey blood from the patient's left ventricle into pumping unit 12. An outflow conduit 13 conveys blood from pumping unit 12 to the patient's aorta. A percutaneous power cable 14 extends from pumping unit 12 outwardly of the patient's body via an incision to a compact control unit 15 worn by patient 10. Control unit 15 is powered by a main battery pack 16 and/or an external AC power supply and an internal backup battery. Control unit 15 includes a commutator circuit for driving a motor within pumping unit 12.

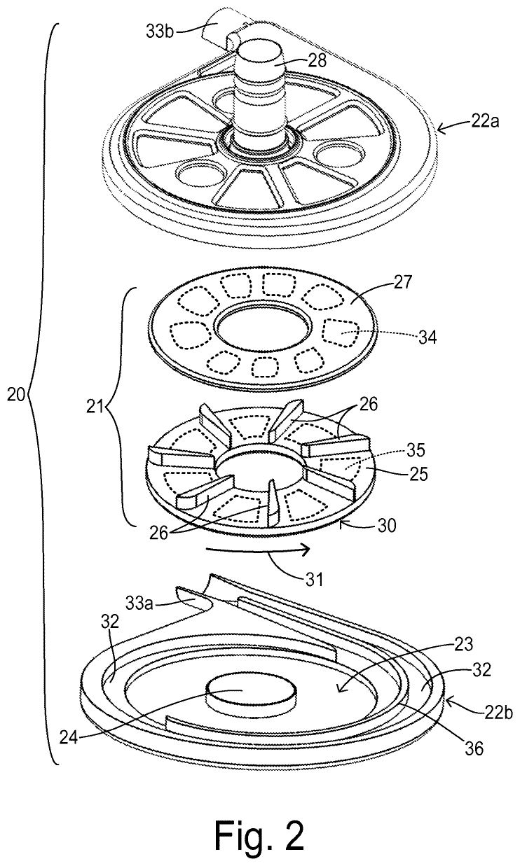

FIG. 2 shows a centrifugal pump unit 20 having an impeller 21 and a pump housing having upper and lower halves 22a and 22b. Impeller 21 is disposed within a pumping chamber 23 over a hub 24. Impeller 21 includes a first plate or disc 25 and a second plate or disc 27 sandwiched over a plurality of vanes 26. Second disc 27 includes a plurality of embedded magnet segments 34 for interacting with a levitating magnetic field created by a levitation magnet structure (not shown) that would be disposed against or incorporated in housing 22a. First disc 25 also contains embedded magnet segments 35 for magnetically coupling with a magnetic field from a motor (not shown) disposed against or incorporated in housing 22b. Housing 22a includes an inlet 28 for receiving blood from a patient's ventricle and distributing it to vanes 26. Impeller 21 is preferably circular and has an outer circumferential edge 30. By rotatably driving impeller 21 in a pumping direction 31, the blood received at an inner edge of impeller 21 is carried to outer circumferential 30 and enters a volute region 32 within pumping chamber 23 at an increased pressure. The pressurized blood flows out from an outlet 33 formed by housing features 33a and 33b. A flow-dividing guide wall 36 may be provided within volute region 32 to help stabilize the overall flow and the forces acting on impeller 21.

FIG. 3 shows a ventricular pressure curve 40 and a ventricular volume curve 41 according to a typical cardiac cycle. A pressure pulse 42 and a volume ejection 43 correspond with a systolic phase 44. A low ventricular pressure 45 and increasing ventricular volume 46 correspond with a diastolic phase 47. The start of systole corresponds with the time that the mitral or tricuspid valve closes, and the start of diastole corresponds with the time that the aortic valve or pulmonary valve closes. Curve 48 shows a pulsatile pump flow in which the pump speed is synchronously varied in order to provide an increased speed during systolic phase 44 and a decreased speed during diastolic phase 47. As explained above, this conventional pulsatile flow does not significantly unload the ventricle at the beginning of systole.

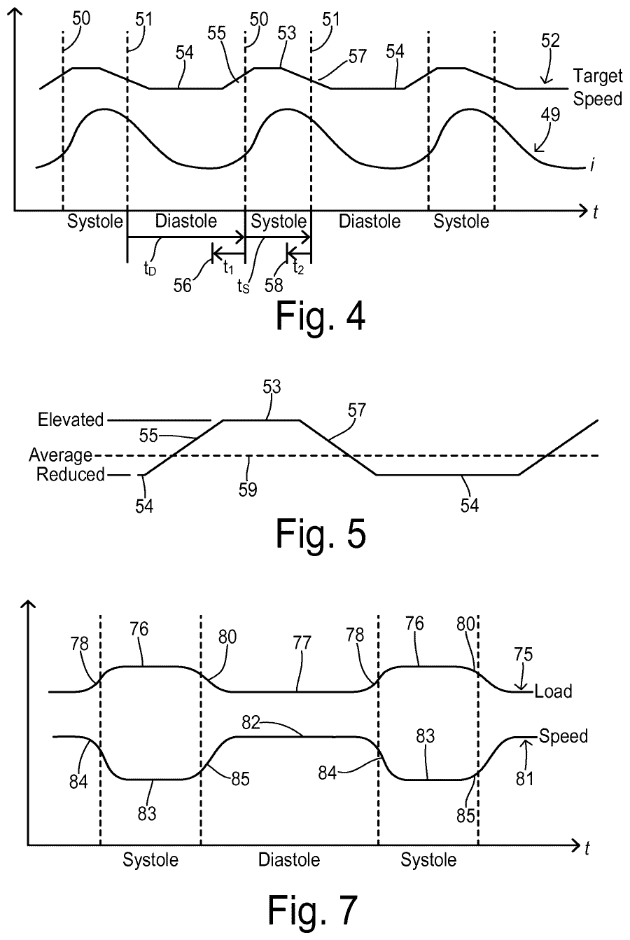

FIGS. 4 and 5 show a first embodiment of the invention for providing a variable speed mode with certain speed changes preceding the beginning of systole 50 and the beginning of diastole 51. An impeller speed curve 52 provides a pulsatile flow between an elevated speed 53 and a reduced speed 54. Curve 49 shows a representative current vector applied to the motor by the motor controller in order to generate a corresponding target speed. Thus, an increase in current generates a speed increase and a decrease in current generates a speed decrease. The target speed includes a ramping up at segment 55 from reduced speed 54 to elevated speed 53, wherein the ramping up begins during diastole. The time of increase and the slope of the increase are configured to provide an increasing flow before systole begins to provide a reduced ventricular pressure that allows a weak ventricle to contract with reduced resistance. The controlled motor current 49 begins to increase at a time 56 which precedes systole by a time period t₁. The beginning of ramping up segment 55 of the speed may lag the current increase but still occurs prior to the beginning of systole 50. Preferably, time 56 is scheduled by the motor controller at time t₁ before the next expected occurrence of systole 50 such that the ramping up begins at a moment between about 50% to about 90% into the diastolic phase. Thus, denoting the length of the diastolic phase as t_{D}, the ratio t₁/t_{D} is preferably between 0.1 and 0.5.

To help avoid collapse of the ventricle toward the end of systole or during diastole, impeller speed 52 preferably ramps down at segment 57 from elevated speed 53 to reduced speed 54. Segment 57 begins during the systolic phase of the cardiac cycle (i.e., before the beginning of diastole 51). For example, current curve 49 starts to ramp down at a time 58 which precedes start of diastole 51 by a time t₂. Preferably, time 58 may be at a moment between about 50% to about 90% into the systolic phase. Thus, denoting the length of the systolic phase as tₛ, the ratio t₁/tₛ is preferably between 0.1 and 0.5.

As shown in FIG. 5, an average speed 59 is maintained as the instantaneous speed varies between elevated speed 53 and reduced speed 54. Average speed 59 may be determined in a conventional manner according to the physiological state of the patient. Offsets from average speed 59 for elevated speed 53 and reduced speed 54 may be substantially constant or may also be determined according to the physiological state of the patient.

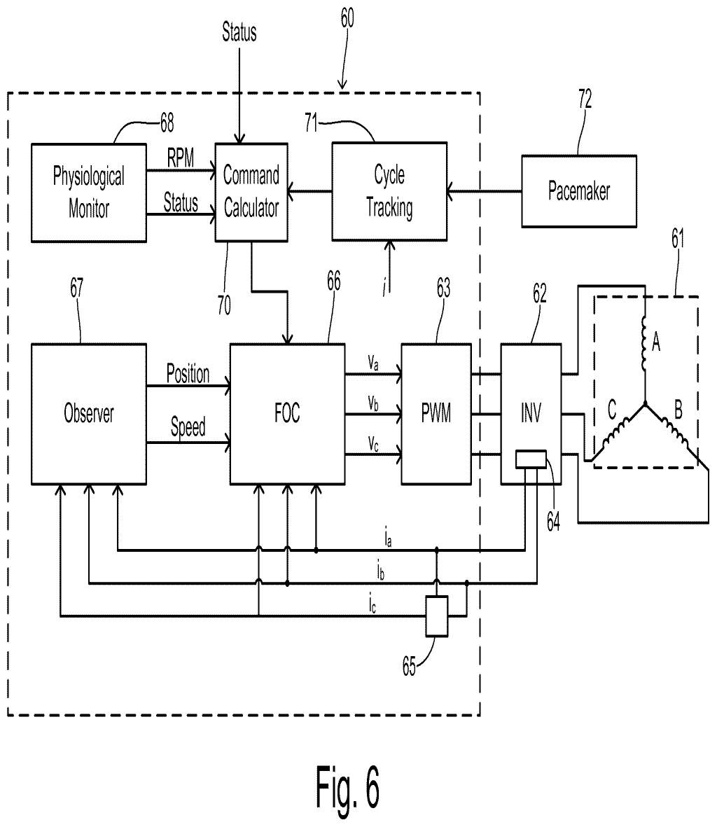

A pump system of the present invention is shown in greater detail in FIG. 6 wherein a controller 60 uses field oriented control (FOC) to supply a multiphase voltage signal to the pump motor which comprises a stator assembly 61 shown as a three-phase stator. Individual phases A, B, and C are driven by an H-bridge inverter 62 functioning as a commutation circuit driven by a pulse width modulator (PWM) circuit 63 in controller 60. A current sensing circuit 64 associated with inverter 62 measures instantaneous phase current in at least two phases providing current signals designated iₐ and i_{b}. A current calculating block 65 receives the two measured currents and calculates a current i_{c} corresponding to the third phase as known in the art. The measured currents are input to an FOC block 66 and to a current observer block 67 which estimates the position and speed of the impeller as known in the art. The impeller position and speed are input to FOC block 66.

An average target speed or rpm for operating the pump is provided by a physiological monitor 68 to FOC block 66. The average rpm may be set by a medical caregiver or may be determined according to an algorithm based on various patient parameters such heart beat. Monitor 68 may also generate a status signal for identifying whether the ventricle is in the initial, highly weakened state or whether a predetermined recovery has been obtained in the strength of the ventricle. The average rpm and the status signal are provided to a speed command calculator 70. The status signal can be used to determine whether or not the variable speed control of the invention should be used to unload the ventricle. The status signal can alternatively be externally provided to calculator 70 (e.g., by a physician via an HMI).

Command calculator 70 is coupled to a cycle tracking block 71 which maintains timing for a cardiac cycle reference. A current signal (e.g., currents iₐ, i_{b}, and i_{c} can be used in order to detect the cardiac cycle from the instantaneous blood flow, for example. More specifically, the controller may identify the heart rate by measuring time between current peaks in the speed control mode. Then the speed decrease can start at a calculated time after the occurrence of a current peak. The speed increase can start at a calculated time after the current minimum value is detected. This calculated time typically depends on the heart rate.

Alternatively, cycle tracking block 71 can be coupled to a pacemaker 72 in the event that the patient is using such a device. Conventional pacemakers have been constructed to continuously generate radio signals that contain information about pulse timing and other data. These sine-wave modulated signals can be received by a special receiver (not shown), where the signals are demodulated, digitized (if necessary), and transferred to cycle tracking block 71. Besides being located near the implanted pacemaker and connected by a cable or wirelessly to the controller (e.g., via BlueTooth), a receiver could be integrated with the controller or the pumping unit.

Based on the reference cycle timing from block 71, command calculator 70 determines an instantaneous speed (or magnitude of the current vector) to be used by FOC block 66. FOC block 66 generates commanded voltage output values Vₐ, V_{b}, and V_{c} which are input to PWM block 63. The Vₐ, V_{b}, and V_{c} commands may also be coupled to observer 67 for use in detecting speed and position (not shown). Thus, the speed is controlled to follow the curves shown in FIGS. 4 and 5.

In one embodiment, the timing of the speed increases and decreases are determined as follows. At a constant pacing rate (i.e., constant beat rate), the time for starting the speed acceleration (e.g., at time 56 in FIG. 4) is:

$${t}_{acc} ( n + 1 ) = {t}_{p} ( n ) + 60 / N - {t}_{1} .$$

where tₚ(n) is the time of occurrence of a pacemaker pulse time signaling the start of the current cardiac cycle; N is the heart (pulse) rate in beat/min set by a pacemaker; and t_{acc}(n+1) is the time to increase the pump speed for the next cardiac cycle.

Similarly, the time to start deceleration (e.g., at a time 58 in FIG. 4) is:

$${t}_{decel} ( n + 1 ) = {t}_{a} ( n + 1 ) + {t}_{s}$$

where tₛ is the duration of systole. Systole typically lasts 30% to 50% of the cardiac cycle 60/N, and within a certain heart rate range it is fairly independent of the heart rate N. For example, for a heart rate N between 60-120 beats/min, tₛ is between 0.30 seconds and 0.25 seconds.

In an alternative embodiment, command calculator 70 and FOC block 66 are configured to operate the motor in a constant current mode (i.e., a constant torque mode). In this mode, the speed changes inversely with the pump load (i.e., the flow rate). Thus, an average speed is determined by the physiological monitor. The motor controller adjusts the current to obtain the desired average speed and to keep the current substantially constant. By keeping a constant current in the face of a load which varies within the cardiac cycle, the impeller speed automatically changes. FIG. 7 shows a load curve 75, wherein the load (i.e., flow rate) is high at 76 during systole and low at 77 during diastole. The load ramps up at 78 before the beginning of systole due to an increase of pressure within the ventricle and a decrease of pressure at the pump outlet (e.g., at the aorta). The load ramps down at 80 during the beginning of diastole.

In the current control mode, the pump flow increases (load increases) in the beginning of systole (at 78) and the speed curve 81 drops to a reduced speed 83. At the end of systole, the flow drops (at 80) and speed increases to an elevated speed 82. Thus, the speed increases and stays relatively high during diastole to help unload the ventricle by pumping out blood at the time it fills the ventricle. This is a natural behavior of the pump in the current control mode.

Either the variable speed control mode using a variable target speed or using the constant current approach of the invention can be combined with the conventional constant speed mode in order to adapt pump performance to the strength level of the patient's ventricle. In particular, the selection between the variable speed mode and the constant speed mode can be determined according to a physiologic capability of the patient. For example, the pump is set to operate in the constant current mode immediately following the implantation when the left ventricle is weak, thereby providing a greater level of ventricle unloading. With the patient's recovery, the pump may be set to operate in the constant speed mode, promoting higher flow pulsatility and a more natural physiologic response to the patient's activities.

What is claimed is:

1. A circulatory support system comprising:
a blood flow inlet, a blood flow outlet, an impeller, and a stator assembly, wherein the stator assembly is configured to receive phase currents used to magnetically rotate the impeller in a pumping direction to pump blood received through the blood flow inlet from a ventricle of a user out through the blood flow outlet to an artery of the user; and
a controller operable to control supply of the phase currents to the stator assembly to operate the stator assembly in a constant current mode to reduce load on the ventricle during ventricular systole.

2. The circulatory support system of claim 1, wherein a flow rate of blood pumped by the impeller increases and a rotational speed of the impeller decrease over an initial portion of ventricular systole during operation of the stator assembly in the constant current mode.

3. The circulatory support system of claim 2, wherein the flow rate of blood pumped by the impeller decreases and the rotational speed of the impeller increases during a final portion of ventricular systole during operation of the stator assembly in the constant current mode.

4. The circulatory support system of claim 3, wherein the rotational speed of the impeller reaches a minimum rotational speed during ventricular systole.

5. The circulatory support system of claim 4, wherein the rotational speed of the impeller reaches a maximum rotational speed during ventricular diastole.

6. The circulatory support system of claim 5, wherein the controller is configured to:
receive an average rotational speed for the impeller over a cardiac cycle of the user, wherein the average rotational speed is specified by a medical caregiver; and
control supply of the phase currents to the stator assembly so that the impeller has the average rotational speed.

7. The circulatory support system of claim 5, wherein:
the controller comprises a physiological monitor configured to set an average rotational speed for the impeller over a cardiac cycle of the user based on a physiological parameter of the user; and
the controller is configured to control supply of the phase currents to the stator assembly so that the impeller has the average rotational speed.

8. The circulatory support system of claim 7, wherein the physiological monitor is configured to set an average rotational speed for the impeller based on a heart rate of the user.

9. The circulatory support system of claim 8, wherein the controller is configured to track an instantaneous rotational speed of the impeller and determine the heart rate of the user based on the instantaneous rotational speed of the impeller.

10. The circulatory support system of claim 7, wherein:
the physiological monitor is configured to generate a status signal indicative of a status of the ventricle of the user; and
the controller is configured to determine whether or not to operate the stator assembly in the constant current mode.

11. A method of operating a ventricular assist device (VAD), the method comprising:
receiving an average rotational speed for an impeller of the VAD over one or more cardiac cycles of a user of the VAD; and
controlling, by a controller, supply of phase currents to a stator assembly of the VAD to operate the stator assembly in a constant current mode to reduce load on a ventricle of the user during ventricular systole and so that the impeller has the average rotational speed.

12. The method of claim 11, wherein a flow rate of blood pumped by the impeller increases and a rotational speed of the impeller decrease over an initial portion of ventricular systole during operation of the stator assembly in the constant current mode.

13. The method of claim 12, wherein the flow rate of blood pumped by the impeller decreases and the rotational speed of the impeller increases during a final portion of ventricular systole during operation of the stator assembly in the constant current mode.

14. The method of claim 13, wherein the rotational speed of the impeller reaches a minimum rotational speed during ventricular systole.

15. The method of claim 14, wherein the rotational speed of the impeller reaches a maximum rotational speed during ventricular diastole.

16. The method of claim 11, comprising determining the average rotational speed for the impeller based on a physiological parameter of the user.

17. The method of claim 11, comprising determining the average rotational speed for the impeller based on a heart rate of the user.

18. The method of claim 17, comprising:
determining an instantaneous rotational speed of the impeller; and
determining the heart rate of the user based on the instantaneous rotational speed of the impeller.

19. The method of claim 11, comprising:
receiving a status signal indicative of a status of the ventricle of the user; and
determining whether or not to operate the stator assembly in the constant current mode.
